(19) United States
(12) Patent Application Publication
(10) Pub. No.: US 2026/0035405 A1
(43) Pub. Date: Feb. 5, 2026

(54) METHOD TO INCREASE ANTIBODY YIELD DURING ION EXCHANGE CHROMATOGRAPHY

(71) Applicant: MORPHOSYS GMBH, Planegg (DE)

(72) Inventors: Daniela DAUBERT, Olching (DE); Dominik GELTINGER, Landshut (DE); Karin FELDERER, Munich (DE)

(21) Appl. No.: 19/363,767

(22) Filed: Oct. 21, 2025

Related U.S. Application Data
(63) Continuation of application No. 17/777,385.

(30) Foreign Application Priority Data
Nov. 22, 2019 (EP) 19210872.8

(51) Int. Cl. C07K 1/18; C07K 16/24

(52) U.S. Cl. CPC C07K 1/18; C07K 16/244

(57) ABSTRACT

The present invention relates to a method for increasing antibody yield during antibody purification from a sample by ion exchange chromatography in flow through mode by pre-conditioning the sample with Tris without the use of NaCl to adjust the conductivity.

### Method to Increase Antibody Yield During Ion Exchange Chromatography

This patent application is a continuation of U.S. application Ser. No. 17/777,385 filed May 17, 2022, which is the U.S. National Stage of International Application No. PCT/EP2020/082820 filed Nov. 20, 2020, which claims the benefit of priority from EP 19210872.8 filed Nov. 22, 2019, teachings of each of which are incorporated herein by reference in their entirety.

### Reference to an Electronic Sequence Listing

The contents of the electronic sequence listing (MOR0087USC1.xml); Size 22,707 bytes; and Date of Creation: Oct. 17, 2025) is herein incorporated by reference in its entirety.

### FIELD OF THE INVENTION

The present disclosure relates to a method for increasing antibody yield during antibody purification from a sample by ion exchange chromatography. The method comprises a multimodal anion-exchange chromatography step in flow-through mode of an antibody sample pre-conditioned with Tris without the use of NaCl to adjust the conductivity prior to the IEX step. The addition of Tris and not only the adjustment of conductivity, improves the yield of the antibody in the flow-through with high monomer content. Further, the present invention is directed toward pharmaceutical compositions comprising an antibody purified with the method described herein.

### BACKGROUND OF THE INVENTION

Monoclonal antibodies (mAbs) are used for therapeutic and diagnostic applications as well as for a broad range of immunochemical techniques within basic research. For use in pharmaceutical applications, therapeutic antibodies have to meet high quality standards. To meet these requirements the goal of every manufacturing and purification process development is therefore to develop a robust, scalable and reliable process that results in a target product with high yield and purity.

During purification the target antibody is to be freed from undesired contaminants, such as host cell proteins (HCPs), nucleic acids, viruses, medium components (e.g. insulin), cell culture additives (e.g. PEG ethers, antifoam) as well as any potentially present aggregated and fragmented product. Antibodies are typically produced by hybridoma or transfected host cells (e.g. CHO, HEK). Therefore, the cellular material has to be removed from the cell culture at the beginning of the purification process. Subsequently, the sample containing the antibody is processed, usually through an affinity chromatography step in bind-elute mode (“capturing”, e.g. by Protein A chromatography for IgG antibodies), followed by virus inactivation, neutralization and depth filtration. To fulfill the quality standards of therapeutic grade products, additional so-called “polishing” steps are included in the purification process. Usually, two or more chromatographic steps are implemented after the “capturing” step to remove residual aggregates and impurities. The impurities generally removed during these steps are process-derived contaminants, such as HCPs, nucleic acids, medium components, leached protein A, endotoxin and the like. Removal of aggregates and impurities is often achieved through use of ion exchange chromatography (IEX) after initial antibody affinity chromatography. In particular, multimodal chromatography (MMC) media are well suited for post-protein A purification of mAbs (Pinto IF et al, Pharm. Bioprocess. (2015) 3 (3), 263-279). The mode of operation also plays an important part for successful multimodal chromatography. In flow-through (FT) mode, for example, the pH of the sample and buffer is selected to modify the charge of the antibody or the chromatography medium such that the antibody will not bind but will rather flow through the column, leaving most impurities bound. The purity of the antibody found in the flow-through fractions can be increased by optimizing conditions such as protein load, pH, and conductivity.

For example, WO2010071208 discloses an antibody purification method by MMC in FT mode utilizing a loading solution containing 20 mM citric acid, pH 6.2 and a specific amino acid selected from arginine, histidine, proline, glutamic acid and citrulline. U.S. Pat. No. 10,023,608 describes the purification of adalimumab, utilizing a MMC step in flow-through mode followed by a HIC step with a sample pre-conditioned with NaCl and Tris base as buffer to achieve the appropriate conductivity (˜15 to 19 mS/cm) and pH (˜ 6.9 to 7.3), respectively of the sample prior to loading the MMC resin. Here conductivity is adjusted with a buffer comprising NaCl. A plethora of various other multi-step chromatography purification methods exists. For instance, WO2005044856 relates to the removal of high-molecular weight aggregates from an antibody sample, using a hydroxyapatite resin optionally in combination with anion exchange chromatography. In preparation for flow-through mode hydroxyapatite chromatography, the antibody sample is adjusted with a load buffer containing 0.2 to 2.5 M NaCl. WO2010048183 relates to the removal of HCP from an antibody sample by consecutive ion exchange at acid pH and HIC chromatography. Prior to the IEX step in FT mode the sample is equilibrated with 20 mM sodium phosphate and 150 mM NaCl or a buffer with 25 mM trolamine and 40 mM NaCl. WO2011090719 describes a method for antibody purification comprising multiple chromatographic steps wherein the low pH eluate from a protein A chromatography is further purified without the need of pH adjustment. WO2012059308 discloses intermediate polishing steps comprising in either order anion exchange chromatography (AEX) chromatography or cation exchange chromatography (CEX) chromatography in flow-through mode. Samples were either diluted with demineralized water up to a final conductivity of 5 mS or adjusted with 50 mM NaH2PO4 in order to adjust pH and conductivity before entering the AEX and CEX column, respectively.

A general drawback of methods with more than one chromatography step is loss of target protein after each particulate step, often resulting in a significant decrease of antibody yield. More steps also mean a longer purification time, which can be detrimental to protein stability and activity. Thus, there is a continuing need to develop methods resulting in an improved yield and at the same time with satisfactory purity. Such methods are of great value for purification processes of therapeutic and diagnostic compounds.

In flow-through mode, appropriate pH and conductivity conditions need to be defined to customize the charge of the target antibody such that the antibody will not bind but will flow through the resin, with the majority of impurities bound to the column. Benefits of flow-through mode are that higher loads can be used and that there are fewer wash and elution steps. Purity of the antibody in the flow-through can be increased by optimizing conditions such as load, pH, salt and conductivity, however predicting optimal conditions is difficult, as the level of contaminants varies between different cell lines. Furthermore, differences in previous purification steps may exist resulting in load samples of varying composition. In general, the prior art (GE Healthcare Instructions 28-9064-05 AA) teaches that for best yield using multimodal Capto adhere in flow-through mode sample load should be high, pH low and conductivity high. For optimal aggregate clearance, pH should be high, while load and conductivity should be low. Aggregate clearance is often less affected by conductivity than Protein A and HCP clearance. For best Protein A and HCP-clearance, pH should be high and conductivity low. Loading conditions will therefore be a compromise between conditions favoring yield and conditions favoring contaminant clearance. Optimal loading conditions will be a balance between load, pH and conductivity. NaCl is a useful salt for conductivity adjustment and widely used because of its cheapness. A change in NaCl concentration and thus conductivity influences the binding strength of charged groups of proteins bound to the ion exchanger. In flow-through mode, a wash step might be used to increase the yield of the target antibody by allowing weakly bound proteins to be collected.

Further purification processes are disclosed e.g. in EP2639239 that relates to a method for removal of protein aggregates from a sample using CEX. There the feed sample is dialyzed into Tris-HCl buffer, pH 7.5, conductivity 3 mS/cm. WO2014196780 relates to a method of removing impurities by sequential use of CEX, a filter and AEX without using affinity chromatography. Before AEX the sample is treated with Tris-HCl and Bis-Tris and adjusted to a conductivity of 1.4 mS/cm. WO2014207763 discloses purification of adalimumab by affinity and hydrophobic interaction chromatography. None of the protein purification methods in the prior art describes Tris base as compound to adjust conductivity.

The technical problem underlying the present application may be seen in the provision of a method for increasing antibody yield during multimodal ion exchange chromatography in FT mode and at the same time maintaining efficient reduction of aggregates and other impurities of the antibody-containing sample. The invention fulfils these needs by providing a method characterized in that the conductivity of the antibody sample to be purified is increased by pre-conditioning the sample with Tris only (i.e. without using NaCl or any other salt) prior to loading the chromatography column in flow-through mode.

### SUMMARY OF THE INVENTION

As described herein, the inventors have discovered that addition of Tris at neutral pH to an antibody sample eluate after initial Protein A capture chromatography prior to loading a mixed-mode anion exchange chromatography resin in flow-through mode surprisingly results in greater antibody yield, compared to performing the steps either without pre-conditioning the sample eluate with Tris (i.e. no increase of conductivity) or by adjusting the conductivity with NaCl.

In certain embodiments, the disclosure provides a method for purifying an antibody from a composition comprising the antibody and aggregates and/or impurities, wherein the method comprises the following steps: a) subjecting the composition to a capture chromatography to produce a capture chromatography eluate; b) adding 2 M Tris, pH 7.1 to the capture eluate in a range of 5 to 20% (v/v); c) subjecting the pre-conditioned eluate from step b) to mixed mode (multimodal) anion exchange chromatography in flow-through mode to generate a mixed mode eluate; d) subjecting the mixed mode eluate to a second mixed mode chromatography in bind-elute mode to generate a second mixed mode eluate; and e) collecting fractions comprising the antibody, wherein the method increases the yield of the antibody.

In certain embodiments, the disclosure provides a method for purifying an antibody from a composition comprising the antibody and aggregates and/or impurities, wherein the method comprises the following steps: a) subjecting the composition to a capture chromatography to produce a capture chromatography eluate; b) adding 2 M Tris, pH 7.1 to the capture eluate in a range of 5 to 20% (v/v); c) subjecting the pre-conditioned eluate from step b) to mixed mode (multimodal) anion exchange chromatography in flow-through mode to generate a mixed mode eluate; d) subjecting the mixed mode eluate to a second mixed mode chromatography in bind-elute mode to generate a second mixed mode eluate; and e) collecting fractions comprising the antibody, wherein the method reduces the amount of aggregates and/or impurities from the composition.

In certain embodiments, the disclosure provides a method for purifying an antibody from a composition comprising the antibody and aggregates and/or impurities, wherein the method comprises the following steps: a) subjecting the composition to a capture chromatography to produce a capture chromatography eluate; b) adding 2 M Tris, pH 7.1 to the capture eluate in a range of 5 to 20% (v/v); c) subjecting the pre-conditioned eluate from step b) to mixed mode (multimodal) anion exchange chromatography in flow-through mode to generate a mixed mode eluate; d) subjecting the mixed mode eluate to a second mixed mode chromatography in bind-elute mode to generate a second mixed mode eluate; and e) collecting fractions comprising the antibody, wherein the method increases the yield of the antibody and reduces the amount of aggregates and/or impurities from the composition.

Certain embodiments of the invention are directed toward methods of purifying anti-IL-17C antibodies, or antigen-binding portions thereof, from a sample to render the antibodies substantially free of host cell proteins (HCPs), leached Protein A, aggregates and other impurities.

In one embodiment, the present disclosure provides a method of purifying IL-17C antibodies that comprises an initial recovery step to remove cells and cellular debris. The recovery step includes one or more centrifugation or depth filtration steps.

In a certain embodiment, the initial recovery sample comprising the antibody is subjected to an affinity chromatography step. Examples include Protein A, Protein G, affinity supports comprising other Fc binding proteins, and affinity supports comprising the antigen against which the antibody of interest was raised. In particular, Protein A is useful for affinity purification of IgG antibodies. In one aspect, a Protein A column is equilibrated with a suitable buffer prior to sample loading. An example of a suitable buffer is PBS, pH 7.0-7.3. Following equilibration, the sample can be loaded onto the column. After loading the column, one or multiple washing steps using, e.g., the equilibrating buffer can be applied. Other washes employing different buffers can be used as well before eluting the column. Elution of the antibody from the affinity column is carried out using an appropriate elution buffer. An example of a suitable elution buffer is 50 mM acetate buffer, pH 3.6. The eluate can be monitored using techniques well known to those skilled in the art. For example, absorbance at OD280 can be measured. The eluted fraction(s) of interest can then be prepared for further steps that usually includes polishing chromatography.

In one embodiment, a low pH adjustment step follows Protein A affinity chromatography. In such embodiment, the Protein A eluate comprising the antibody is subjected to a pH adjustment to a pH of about 2.5 to about 3.5 using 1 M acetic acid for reduction and/or inactivation of pH-sensitive viruses that may be contaminating the sample. In a particular embodiment, the affinity eluate is adjusted to a pH of 3 with 1 M acetic acid. After a defined incubation period the solution is then neutralized to a pH between about 6.5 and about 7.5. In one embodiment, the pH neutralization may be accomplished using 1 M Tris, pH 9.5 buffer. In one embodiment, depth filtration follows virus inactivation (i.e. low pH adjustment) and neutralization.

In certain embodiments, ion exchange chromatography follows affinity chromatography. In other embodiments, ion exchange follows the low pH adjustment step. In a preferred embodiment, ion exchange follows depth filtration after a virus inactivation step. The ion exchange step can be either cation or anion exchange. This step can be a single ion exchange procedure or can include multiple ion exchange steps in a sequential combination such as a cation exchange followed by an anion exchange or vice versa.

In certain embodiments, Capto adhere ImpRes (GE Healthcare) as a strong anion exchange chromatography resin with multimodal functionality, may be used as a polishing step. In one embodiment, this step is performed in flow-through mode under conditions wherein the antibody to be purified does not bind the ion exchange resin, whereas main contaminants such as DNA, RNA, host cell proteins, aggregates and viruses do bind and are thus efficiently separated.

In one aspect, the antibody sample (e.g. affinity chromatography eluate, filtrate after depth filtration) is prepared for ion exchange chromatography by adjusting the pH and ionic strength or conductivity of the sample.

In a preferred embodiment, the method for purifying an antibody comprises the steps of:
a. providing a sample comprising an antibody;
b. adjusting the conductivity of the sample;
c. processing the adjusted sample by ion exchange chromatography in flow-through mode and
d. collecting the flow-through comprising the antibody, wherein the conductivity of the sample in step b) is adjusted with Tris, preferably with 2 M Tris, pH 7.1.

In another embodiment, the method for increasing the yield of an antibody comprises the steps of:
a. providing a sample comprising an antibody;
b adjusting the conductivity of the sample;
c. processing the adjusted sample by ion exchange chromatography in flow-through mode and
d. collecting the flow-through comprising the antibody, wherein the conductivity of the sample in step b) is adjusted with Tris, preferably with 2 M Tris, PH 7.1.

In one aspect, the method for purifying an antibody comprises the steps of:
a. providing a sample comprising an antibody;
b. adjusting the conductivity of the sample;
c. processing the adjusted sample by ion exchange chromatography in flow-through mode and
d. collecting the flow-through comprising the antibody, wherein the conductivity of the sample in step b) is adjusted with Tris to a conductivity of about 10 to about 50 mS/cm. Preferably, conductivity is adjusted to about 10 to about 30 mS/cm. In certain embodiments, the conductivity of the sample after pre-conditioning with Tris in step b) is at least 10 mS/cm, at least 12 mS/cm, at least 14 mS/cm, at least 15 mS/cm. In certain embodiments, the conductivity of the antibody sample after pre-conditioning with Tris in step b) is in the range of about 10 mS/cm to about 30 mS/cm, in the range of about 12 mS/cm to about 28 mS/cm, in the range of about 14 mS/cm to about 26 mS/cm, in the range of about 15 mS/cm to about 25 mS/cm before loading the sample onto the IEX resin. Importantly, adjustment of conductivity is carried out using Tris only.

In one aspect, the method for increasing the yield of an antibody comprises the steps of:
a. providing a sample comprising an antibody;
b. adjusting the conductivity of the sample;
c. processing the adjusted sample by ion exchange chromatography in flow-through mode and
d. collecting the flow-through comprising the antibody, wherein the conductivity of the sample in step b) is adjusted with Tris to a conductivity of about 10 to about 50 mS/cm. Preferably, conductivity is adjusted to about 10 to about 30 mS/cm. In certain embodiments, the conductivity of the sample after pre-conditioning with Tris in step b) is at least 10 mS/cm, at least 12 mS/cm, at least 14 mS/cm, at least 15 mS/cm. In certain embodiments, the conductivity of the antibody sample after pre-conditioning with Tris in step b) is in the range of about 10 mS/cm to about 30 mS/cm, in the range of about 12 mS/cm to about 28 mS/cm, in the range of about 14 mS/cm to about 26 mS/cm, in the range of about 15 mS/cm to about 25 mS/cm before loading the sample onto the IEX resin. Importantly, adjustment of conductivity is carried out using Tris only.

In one embodiment, the provided sample comprising an antibody is obtained after Protein A chromatography, virus inactivation, neutralization and depth filtration, followed by adjusting the conductivity of the sample and then processing the adjusted sample through a first polishing step comprising ion exchange chromatography in flow-through mode, followed by a second polishing step comprising a multimodal cation exchange chromatography step in bind-elute mode. The second mixed mode chromatography carried out in bind and elute mode can apply gradient elution.

In certain embodiments, the ion exchange sample is subjected to an intermediate filtration step, either prior to the first ion exchange step, between the two ion exchange steps, or both. In certain aspects, this filtration step comprises capture ultrafiltration/diafiltration (“UF/DF”). Among other things, such filtration facilitates the concentration and buffer exchange of antibodies and antigen-binding portions thereof.

In one embodiment, the antibody to be purified is a monoclonal antibody.

### BRIEF DESCRIPTION OF THE FIGURES

FIG. 1 Antibody purifications may employ 1-, 2—, or 3-step procedures. Numbers refer to the steps. Typical yield and purity expectations are indicated. AC=affinity chromatography; SEC=size exclusion chromatography; IEX=ion exchange chromatography; CIEX=cation exchange chromatography; AIEX=anion exchange chromatography.

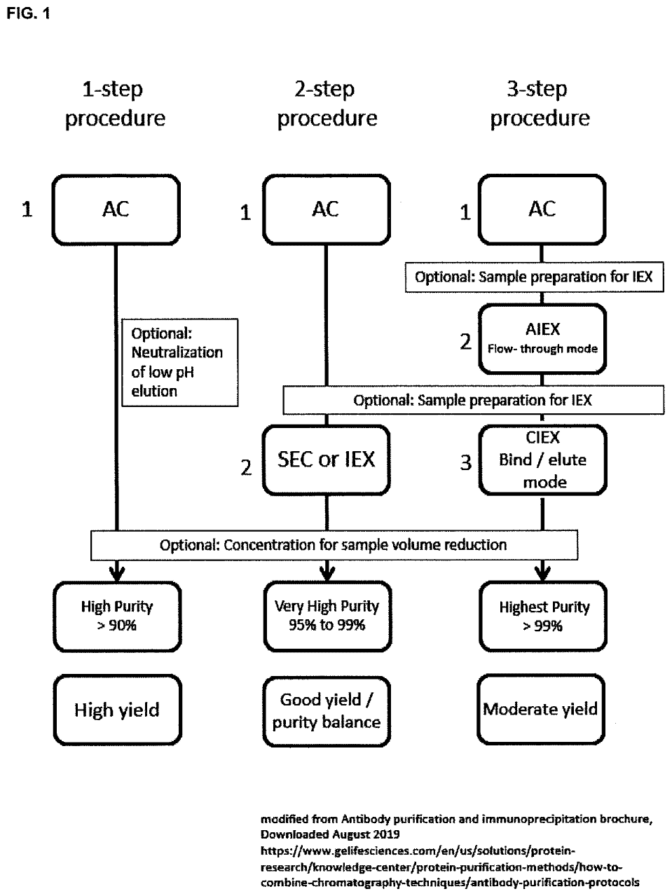

FIG. 2 Representative flow-through elution chromatograms of samples comprising an antibody with a heavy chain of SEQ ID NO.: 10 and a light chain of SEQ ID NO.: 9 pre-conditioned with or without Tris and purified by multimodal AIEX. Pre-conditioning: (1) no Tris addition, conductivity: 8.2 [mS/cm], (2) 5% (v/v) Tris addition, conductivity 13.6 mS/cm, (3) 10% (v/v) Tris addition, conductivity 17.2 mS/cm, (4) 20% (v/v) Tris addition, conductivity 25.5 mS/cm. FT: Flow through. CIP: cleaning in place.

### DETAILED DESCRIPTION OF THE INVENTION

### Protein Purification by Chromatography

Therapeutic antibody manufacturing is typically divided into i) upstream processing (USP), including production of the antibody protein; ii) downstream processing (DSP), comprising yield of the antibody in a pure form by purification; and iii) final processing to gain product integrity and safety. Typically, the first step of a downstream purification process, following the production phase, involves clarification of the harvested cell culture mixture where one or more of steps of precipitation, flocculation, (depth) filtration and/or centrifugation can be used to separate the desired antibody from cells, cellular debris, and other contaminants. Downstream purification typically includes one or more (orthogonal) chromatographic separation steps based on e.g. affinity, ion-exchange, hydrophobic interaction, hydroxyapatite, chromatofocusing, gel filtration and reverse phase to efficiently remove process and product related impurities. These contaminants include but are not limited to HCPs, leached protein A, product isoforms, high molecular weight (HMW), low molecular weight (LMW), and clipped or degraded product.

Affinity chromatography refers to the use of a compound that specifically interacts with a desired target protein to be purified. Usually, the compound is immobilized on a resin for the purpose of isolating, purifying, or removing the desired target product. For antibody purification, for example, affinity resins include Protein A obtained from Staphylococcus aureus, Protein G from Streptococcus sp., Protein L from Peptostreptococcus magnus, and recombinant or synthetic versions or peptides of such. These resins include MAbSelect™ (GE Healthcare), Prosep A® (Millipore) and others. For laboratory scale applications, a one-step affinity purification generally achieves satisfactory purity. Protein A chromatography, for example, as the most widely used affinity purification to capture lgG antibodies supports purity of >95% with excellent recovery due to its high specificity to the Fc part of IgGs. Other examples of well-established purification methods include thiophilic adsorption, hydrophobic interaction or aromatic adsorption chromatography, metal chelate affinity chromatography, and size exclusion chromatography. (Vijayalakshmi, M. A., Appl. Biochem. Biotech. 75 (1998) 93-102).

Further removal of aggregates/impurities can be achieved by a combination of one or two further orthogonal chromatographic steps that may include hydroxyapatite, hydrophobic interaction (HIC), and ion exchange chromatography (IEX, e.g. cation exchange (CEX), anion exchange (AEX), or mixed-mode exchange). At manufacturing scale, removal of aggregates and impurities is often achieved through use of IEX after initial antibody affinity chromatography. Commercial multimodal ion exchangers such as Capto MMC and Capto adhere, as well as Capto MMC ImpRes and Capto adhere ImpRes (all from GE Healthcare) can be used for removal of contaminants downstream of the initial affinity capture. IEX separates proteins with differences in surface charge to give a high-resolution separation with high sample loading capacity. This separation is based on reversible electrostatic interactions between a charged protein (i.e. charged amino acid side chains) and an oppositely charged chromatography medium. AEX involves purification of proteins on a resin with positively charged functional groups (e.g. strong anion exchangers with quaternary amine group, or weak anion exchanger with secondary amine group). CEX involves purification of proteins on a resin with negatively charged functional groups (e.g. strong cation exchangers with sulfite groups, or weak cation exchangers with carboxylate anions). Both, AEX and CEX, have been demonstrated to be effective in removing not only aggregates but also other impurities during production scale processes. Each chromatography step, either cation exchange or anion exchange, can be performed in bind and elute or flow-through mode, depending upon the physicochemical properties of the target protein and impurities. Protein molecules vary considerably in their charge properties and exhibit different degrees of interaction with charged chromatography media according to differences in their overall charge, charge density and surface charge distribution. For example, monoclonal antibodies comprise ionizable groups such as carboxyl groups and amino groups. The charge of these groups will depend on the pH. Therefore, depending on an antibody's isoelectric point (pl) the charge of a protein molecule can be manipulated by exposing the bulk product to different pH conditions. Monoclonal IgG1 antibodies typically have basic pls of around 7-9. In flow-through mode, appropriate pH and conductivity conditions need to be defined to customize the charge of the target antibody such that the antibody will not bind but will flow through the resin, with the majority of impurities bound to the column. AEX chromatography is often run in flow-through mode at neutral to slightly basic pH for removal of impurities such as viruses and DNA, which are expected to bind to the resin while the product is collected in the non-bound fraction. As the mode of separation of an AEX chromatography resin is based on electrostatic interactions factors such as conductivity (controlled by salt concentration) also influence the capability of DNA, host cell protein, aggregates and other impurity clearance in AEX in FT mode.

The essential core of the present invention is that the conductivity of the sample is adjusted with Tris only. Surprisingly, it was found that the addition of Tris, and not only the adjustment of conductivity (e.g. with NaCl), improves the yield of multimodal AEX chromatography in FT mode. In a head to head comparison of a sample load adjusted with Tris only and a sample load adjusted with NaCl to the same conductivity, it was found that with addition of Tris only, antibody yield after MMC in FT mode is about 5% higher and monomer content slightly lower, but still within the specification.

### EMBODIMENTS

In one embodiment, the disclosure relates to a method for purifying an antibody comprising the steps of:
a. providing a sample with a first pH comprising an antibody;
b. adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody, wherein pH and conductivity of the sample in step c) is adjusted with Tris.

In another embodiment, the disclosure relates to a method for increasing the yield of an antibody comprising the steps of:
a. providing a sample with a first pH comprising an antibody;
b adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody, wherein pH and conductivity of the sample in step c) is adjusted with Tris.

In a certain embodiment, the disclosure relates to a method for purifying an antibody comprising the steps of:
a. providing a sample with a first pH comprising an antibody;
b. adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody, wherein pH and conductivity of the sample in step c) is adjusted with Tris to a conductivity of at least 10 mS/cm.

In a certain embodiment, the disclosure relates to a method for purifying an antibody comprising the steps of:
a. providing a sample with a first pH comprising an antibody;
b. adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody, wherein pH and conductivity of the sample in step c) is adjusted with Tris to a conductivity between 10 and 50 mS/cm. Preferably, the conductivity is adjusted to 15 mS/cm.

In another embodiment, the disclosure relates to a method for increasing the yield of an antibody comprising the steps of:
a. providing a sample with a first pH comprising an antibody;
b. adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody, wherein pH and conductivity of the sample in step c) is adjusted with Tris to a conductivity between 10 and 50 mS/cm. Preferably, the conductivity is adjusted to 15 mS/cm.

In another embodiment, the disclosure relates to a method for purifying an antibody comprising the steps of:
a. providing a sample with a first pH comprising an antibody;
b. adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody,
   wherein pH and conductivity of the sample in step c) is adjusted with Tris to a conductivity between 10 and 30 mS/cm and a third pH of about 6.5 to 7.5. Preferably, the conductivity is adjusted to 15 mS/cm and the pH to about 7.1.

In another embodiment, the disclosure relates to a method for increasing the yield of an antibody comprising the steps of:
a. providing a sample with a first pH comprising an antibody;
b. adjusting the first pH of the sample to a second pH;
c. adjusting the conductivity of the sample and the second pH to a third pH;
d. processing the adjusted sample by ion exchange chromatography in flow-through mode and
e. collecting the flow-through comprising the antibody,
   wherein pH and conductivity of the sample in step c) is adjusted with Tris to a conductivity between 10 and 30 mS/cm and a third pH of about 6.5 to 7.5. Preferably, the conductivity is adjusted to 15 mS/cm and the pH to about 7.1.

In preferred embodiments, the sample in step a) is an affinity chromatography eluate obtained after an affinity chromatography step. Most preferably the sample in step a) is a Protein A chromatography eluate with a first pH of about 3 to about 4, ideally with a first pH of 3.6. Non-limiting examples of affinity chromatography supports include, but are not limited to Protein A, Protein G, Protein L and affinity supports comprising the antigen against which the antibody of interest was raised. In certain aspects, the Protein A chromatography resin is selected from ProSep Ultra Plus, MabSelect SuRe™, or Amsphere Protein A™ resins. Prior to sample loading the affinity column is equilibrated with a suitable buffer (e.g. PBS, pH 7.0-7.3). After the sample is loaded onto the column the column is washed one or multiple times using a suitable washing buffer (e.g. PBS, pH 7.0-7.3). The antibody bound to the affinity support can then be eluted using an appropriate elution buffer (e.g. Na-Acetate buffer, pH 3.6).

In other embodiments, the disclosure relates to a method according to any of the preceding embodiments, wherein the second pH of step b) is adjusted to a pH of about 5.2 to about 5.6. Preferably, the second pH is adjusted to pH 5.5.

In preferred embodiments, mixed mode or mixed modal or multimodal (“MM”) chromatography may be used as ion exchange chromatography in step d). This mixed mode step can feature either cation or anion exchange or a combination of both. This step can be based on a single type of ion exchanger mixed mode procedure or can include multiple ion exchanger mixed mode steps such as a cation exchange mixed mode step followed by an anion exchange mixed mode step or vice versa. Chromatographic mediums for MM chromatography include, among others, mixtures of the following: anion exchange medium, cation exchange medium, hydrophobic interaction medium, hydrophilic interaction medium, hydrogen bonding, pi-pi bonding, and metal affinity. In some embodiments, an MM chromatographic medium with at least an ion exchange medium, such as an anion exchange medium or a cation exchange medium is used in the MM chromatography. A suitable cation exchange column is a column whose stationary phase comprises anionic groups. An example of such a column is a Capto MMC™, Capto MMC™ ImpRes (GE Healthcare), Nuvia™ cPrime™ (Biorad). In one embodiment, the cation exchange mixed mode chromatography comprises N-benzyl-n-methyl ethanolamine. In another aspect, a suitable anion exchange column is a column whose stationary phase comprises cationic groups. In one embodiment, the mixed mode chromatography is a Capto™ Adhere chromatography or a Capto™ Adhere ImpRes chromatography (GE Healthcare). In one embodiment, the first mixed mode chromatography is carried out in flow-through mode. Prior to loading the sample (e.g. affinity eluate) onto the mixed mode column, the column can be equilibrated using a suitable buffer.

In other embodiments, the antibody sample (e.g. an affinity chromatography eluate) is prepared for the mixed mode step by adjusting the load, pH, conductivity and ionic strength of the sample.

In one embodiment, the disclosure relates to a method for purifying an antibody comprising the steps of:
a. providing a sample with a first pH comprising an antibody;
b. adjusting the first pH of the sample to a second pH;
c. adjusting the conductivity of the sample and the second pH to a third pH and the load density;
d. processing the adjusted sample by ion exchange chromatography in flow-through mode and
e. collecting the flow-through comprising the antibody,
   wherein the conductivity of the sample in step c) is adjusted to a conductivity between 10 and 30 mS/cm and a third pH of about 6.5 to 7.5 with a load density of about 10 to about 200 g/L. Preferably, the conductivity is adjusted to 15 mS/cm and the third pH is adjusted to pH 7.1 with a load density from 20 to 40 g/L.

In another embodiment, the disclosure relates to a method for increasing the yield of an antibody comprising the steps of:
a. providing a sample with a first pH comprising an antibody;
b. adjusting the first pH of the sample to a second pH;
c. adjusting the conductivity of the sample and the second pH to a third pH and the load density;
d. processing the adjusted sample by ion exchange chromatography in flow-through mode and
e. collecting the flow-through comprising the antibody,
   wherein the conductivity of the sample in step c) is adjusted to a conductivity between 10 and 30 mS/cm and a third pH of about 6.5 to 7.5 with a load density of about 10 to about 200 g/L. Preferably, the conductivity is adjusted to 15 mS/cm and the third pH is adjusted to pH 7.1 with a load density from 20 to 40 g/L.

In one embodiment, the antibody to be purified is applied in a solution with a conductivity of more than 10 mS/cm onto the multimodal anion exchange chromatography resin. In another embodiment, the antibody is applied in a solution with a conductivity in the range of about 10 mS/cm to about 30 mS/cm. In some embodiments, the antibody is applied in a solution with a conductivity of about 15 mS/cm onto the multimodal anion exchange chromatography resin.

In one aspect, the antibody sample is applied in the range from about 1 to 300 g, about 5 to 200 g, about 10 to 100 g, about 20 to 50 g, 20 to 40 g per liter of resin material in the multimodal anion exchange chromatography step.

In one embodiment, the present disclosure refers to a method for purifying an antibody specific for IL-17C by multimodal anion exchange (MM-AIEX) chromatography in flow-through mode, comprising the following steps:
a. providing an affinity chromatography (AC) eluate with a first pH comprising the anti-IL 17C antibody;
b. adjusting the first pH of the AC eluate to a second pH;
c. adjusting the conductivity of the eluate and the second pH to a third pH;
d. processing the adjusted eluate by ion exchange chromatography in flow-through mode and
e. collecting the flow-through comprising the anti-IL-17C antibody,
   wherein pH and conductivity of the eluate in step c) is adjusted with 2 M Tris to a Tris concentration of 5% (v/v), 10% (v/v), 15% (v/v), 20% (v/V) or in the range of 5% (v/v) to 20% (v/v) and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for purifying an antibody specific for IL-17C by multimodal anion exchange (MM-AIEX) chromatography in flow-through mode, comprising the following steps:
a. providing an affinity chromatography (AC) eluate with a first pH of about 3 to about 4 comprising an anti-IL-17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v), 20% (v/v) or in the range of 5% (v/v) to 20% (v/v) and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for purifying an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v), 20% (v/v) or in the range of 5% (v/v) to 20% (v/v) and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a heavy chain and a light chain that have at least 90%, at least 91%, at least 92%, at least 93%, at least 94%, at least 95%, at least 96%, at least 97%, at least 98% or at least 99% sequence identity to the heavy chain of SEQ ID No.: 10 and the light chain of SEQ ID No.: 9.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH comprising an snti-IL 17C antibody;
b. adjusting the first pH of the AC eluate to a second pH;
c. adjusting the conductivity of the eluate and the second pH to a third pH;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/V), 20% (v/v) or in the range of 5% (v/v) to 20% (v/v) and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v) or 20% (v/v) or in the range of 5% (v/v) to 20% (v/v) and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v) or 20% (v/v) or in the range of 5% (v/v) to 20% (v/v) and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a heavy chain and a light chain that have at least 90%, at least 91%, at least 92%, at least 93%, at least 94%, at least 95%, at least 96%, at least 97%, at least 98% or at least 99% sequence identity to the heavy chain of SEQ ID No.: 10 and the light chain of SEQ ID No.: 9.

In preferred embodiments, the adjustment of pH and conductivity of the antibody sample in step c) with Tris leads to an increase in yield of the antibody in the flow-through after multimodal anion exchange chromatography. In an embodiment adjustment with 2 M Tris, pH 7.1 to a concentration of 5% (v/v) leads to an antibody yield of more than or equal to 70%, 71%, 72%, 73%, 74%, 75%, 76%, 77%, 78%, 79%, 80%, 81%, 82%, 83%, 84%, 85%, 86%, 87%, 88%, 89%, 90% after the multimodal anion exchange chromatography step. In another embodiment adjustment with 2 M Tris, PH 7.1 to a concentration of 10% (v/v) leads to an antibody yield of more than or equal to 70%, 71%, 72%, 73%, 74%, 75%, 76%, 77%, 78%, 79%, 80%, 81%, 82%, 83%, 84%, 85%, 86%, 87%, 88%, 89%, 90% after the multimodal anion exchange chromatography step. In another embodiment adjustment with of 2 M Tris, PH 7.1 to a concentration of 15% (v/v) leads to an antibody yield of more than or equal to 70%, 71%, 72%, 73%, 74%, 75%, 76%, 77%, 78%, 79%, 80%, 81%, 82%, 83%, 84%, 85%, 86%, 87%, 88%, 89%, 90% after the multimodal anion exchange chromatography step. In yet another embodiment adjustment with 2 M Tris, pH 7.1 to a concentration of 20% (v/v) leads to an antibody yield of more than or equal to 70%, 71%, 72%, 73%, 74%, 75%, 76%, 77%, 78%, 79%, 80%, 81%, 82%, 83%, 84%, 85%, 86%, 87%, 88%, 89%, 90% after the multimodal anion exchange chromatography step.

In one embodiment, the antibody or antibody fragment to be purified is a human, humanized or chimeric antibody or antibody fragment.

In certain embodiments, the antibody to be purified is an IgA1, IgA2, IgD, IgE, IgG1, IgG2, IgG3, IgG4, or IgM isotype antibody.

In preferred embodiments, the antibody to be purified is of the lgG isotype or variants thereof. More preferably, the antibody is an IgG1 antibody.

In one embodiment, the present disclosure refers to the purification of an antibody specific for IL-17C. In other embodiments the mAb to be purified shares more than or equal to 80%, 85%, 90%, 91%, 92%, 93%, 94%, 95%, 96%, 97%, 98% or 99% identity in the CDRs compared to the CDRs of SEQ ID NO.: 1, SEQ ID NO.: 2, SEQ ID NO.: 3 and SEQ ID NO.: 4, SEQ ID NO.: 5, and SEQ ID NO.: 6.

Antibody preparations to which the invention can be applied can include unpurified or partially purified antibodies from natural, synthetic, or recombinant sources. The antibody sample may be cell culture material, for example, solubilized cells and cell culture supernatant. In certain embodiments, it is a clarified cell culture harvest. The methods of the invention can be used as a polishing step to purify an antibody from any mixture containing the antibody. For example, such a mixture can be a Protein A eluate.

Further, the present invention is directed toward pharmaceutical compositions comprising one or more antibodies purified by a method described herein.

The purity of the antibodies of interest in the resultant sample product can be analyzed using methods well known to those skilled in the art, e.g., size-exclusion chromatography, Poros™ A HPLC Assay, HCP ELISA, Protein A ELISA, and western blot analysis.

In a preferred embodiment, the method provided herein results in a purified antibody having a SEC monomer content of equal to or more than 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9%. In another embodiment, the purified protein has a SEC monomer content of 100%.

In another embodiment, the method provided herein results in a purified antibody with a yield of equal to or more than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH comprising an anti-IL17C antibody;
b. adjusting the first pH of the AC eluate to a second pH;
c. adjusting the conductivity of the eluate and the second pH to a third pH;
d. processing the adjusted eluate by ion exchange chromatography in flow-through mode and
e. collecting the flow-through comprising the antibody,
   wherein pH and conductivity of the eluate in step c) is adjusted with Tris and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL 17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a heavy chain and a light chain that have at least 90%, at least 91%, at least 92%, at least 93%, at least 94%, at least 95%, at least 96%, at least 97%, at least 98% or at least 99% sequence identity to the heavy chain of SEQ ID No.: 10 and the light chain of SEQ ID No.: 9.

In another embodiment, the present disclosure refers to a method for the purification of an antibody specific for IL-17C by multimodal anion exchange (MM-AIEX) chromatography in flow-through mode, comprising the following steps:
a. providing an affinity chromatography (AC) eluate with a first pH comprising an antibody;
b. adjusting the first pH of the AC eluate to a second pH;
c. adjusting the conductivity of the eluate and the second pH to a third pH;
d. processing the adjusted eluate by ion exchange chromatography in flow-through mode and
e. collecting the flow-through comprising the antibody,
   wherein pH and conductivity of the eluate in step c) is adjusted with Tris and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for the purification of an antibody specific for IL-17C by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for the purification of an antibody specific for IL-17C by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a heavy chain and a light chain that have at least 90%, at least 91%, at least 92%, at least 93%, at least 94%, at least 95%, at least 96%, at least 97%, at least 98% or at least 99% sequence identity to the heavy chain of SEQ ID No.: 10 and the light chain of SEQ ID No.: 9.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH comprising an antibody;
b. adjusting the first pH of the AC eluate to a second pH;
c. adjusting the conductivity of the eluate and the second pH to a third pH;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a heavy chain and a light chain that have at least 90%, at least 91%, at least 92%, at least 93%, at least 94%, at least 95%, at least 96%, at least 97%, at least 98% or at least 99% sequence identity to the heavy chain of SEQ ID No.: 10 and the light chain of SEQ ID No.: 9.

In another embodiment, the present disclosure refers to a method for the purification of an antibody specific for IL-17C during purification by multimodal anion exchange (MM-AIEX) chromatography in flow-through mode, comprising the following steps:
a. providing an affinity chromatography (AC) eluate with a first pH comprising an antibody;
b. adjusting the first pH of the AC eluate to a second pH;
c. adjusting the conductivity of the eluate and the second pH to a third pH;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for the purification of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a variable heavy chain and a variable light chain that have at least 90%, at least 91%, at least 92%, at least 93%, at least 94%, at least 95%, at least 96%, at least 97%, at least 98% or at least 99% sequence identity to the variable heavy chain of SEQ ID No.: 8 and the variable light chain of SEQ ID No.: 7.

In another embodiment, the present disclosure refers to a method for the purification of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody comprises a HCDR1 region comprising the amino acid sequence of SEQ ID No.: 1, a HCDR2 region comprising the amino acid sequence of SEQ ID No.: 2, a HCDR3 region comprising the amino acid sequence of SEQ ID No.: 3, a LCDR1 region comprising the amino acid sequence of SEQ ID No.: 4, a LCDR2 region comprising the amino acid sequence of SEQ ID No.: 5 and a LCDR3 region comprising the amino acid sequence of SEQ ID No.: 6 and a heavy chain and a light chain that have at least 90%, at least 91%, at least 92%, at least 93%, at least 94%, at least 95%, at least 96%, at least 97%, at least 98% or at least 99% sequence identity to the heavy chain of SEQ ID No.: 10 and the light chain of SEQ ID No.: 9.

In another embodiment, the present disclosure refers to a method for increasing the yield of an antibody during purification by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v) or 20% (v/v) or in the range of 5% (v/V) to 20% (v/v) wherein said antibody has a heavy chain of SEQ ID No.: 10 and a light chain of SEQ ID No.: 9.

In another embodiment, the present disclosure refers to a method for purification of an antibody by multimodal anion exchange (MM-AIEX) chromatography in flow-through mode, comprising the following steps:
a. providing an affinity chromatography (AC) eluate with a first pH of about 3 to about 4 comprising an antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v) or 20% (v/v) or in the range of 5% (v/v) to 20% (v/v) wherein said antibody has a heavy chain of SEQ ID No.: 10 and a light chain of SEQ ID No.: 9.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL 17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody has a heavy chain of SEQ ID No.: 10 and a light chain of SEQ ID No.: 9.

In another embodiment, the present disclosure refers to a method for the purification of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with Tris in the absence of NaCl and wherein said antibody has a heavy chain of SEQ ID No.: 10 and a light chain of SEQ ID No.: 9.

In another embodiment, the present disclosure refers to a method for increasing the yield of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v) or 20% (v/v) or in the range of 5% (v/v) to 20% (v/v) in the absence of NaCl and wherein said antibody has a heavy chain of SEQ ID No.: 10 and a light chain of SEQ ID No.: 9.

In another embodiment, the present disclosure refers to a method for the purification of an antibody specific for IL-17C during purification by multimodal anion exchange (MM-AIEX) chromatography in flow-through mode, comprising the following steps:
a. providing an affinity chromatography (AC) eluate with a first pH of about 3 to about 4 comprising an anti-IL17C antibody;
b. adjusting the first pH of the AC eluate to a second pH of about 5.2 to about 5.6, preferably 5.5;
c. adjusting the conductivity of the eluate to a conductivity between 10 and 30 mS/cm, preferably 15 mS/cm and the second pH to a third pH of about 7.1;
d. processing the adjusted eluate by ion exchange chromatography in flow-through mode and
e. collecting the flow-through comprising the antibody,
   wherein pH and conductivity of the eluate in step c) is adjusted with 2 M Tris to a Tris concentration of 5% (v/v), 10% (v/v), 15% (v/v) or 20% (v/v) or in the range of 5% (v/v) to 20% (v/v) in the absence of NaCl and wherein said antibody has a heavy chain of SEQ ID No.: 10 and a light chain of SEQ ID No.: 9.

In other embodiments, conductivity is adjusted to at least 10 mS/cm, to a range of 10 to 50 mS/cm, to a range of 10 to 30 mS/cm, 11 to 30 mS/cm, 12 to 30 mS/cm, 13 to 30 mS/cm, 10 to 29 mS/cm, 10 to 28 mS/cm, 10 to 27 mS/cm, 10 to 26 mS/cm, 11 to 29 mS/cm, 11 to 28 mS/cm, 11 to 27 mS/cm, 11 to 26 mS/cm, 12 to 29 mS/cm, 12 to 28 mS/cm, 12 to 27 mS/cm, 12 to 26 mS/cm, 13 to 29 mS/cm, 13 to 28 mS/cm, 13 to 27 mS/cm, 13 to 26 mS/cm, or 13 to 25 mS/cm.

### Definitions

The term “protein” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As used herein, a “peptide,” a “peptide fragment,” a polypeptide “,” an “amino acid chain,” an “amino acid sequence,” or any other term used to refer to a chain or chains of two or more amino acids, are generically included in the definition of a “protein,” even though each of these terms can have a more specific meaning. The term “protein” can be used instead of, or interchangeably with any of these terms. The term further includes proteins, which have undergone post-translational or post-synthesis modifications, for example, glycosylation, acetylation, phosphorylation, or amidation.

A “buffer” is a solution that resists changes in pH by the action of its acid-base conjugate components. Various buffers, which can be employed depending, for example, on the desired pH of the buffer are described in Buffers. A Guide for the Preparation and Use of Buffers in Biological Systems, Gueffroy, D., ed. Calbiochem Corporation (1975). Non-limiting examples of buffers that will control the pH in this range include MES, MOPS, MOPSO, Tris, HEPES, phosphate, acetate, citrate, succinate, and ammonium buffers, as well as combinations of these.

“Tris” or tris(hydroxymethyl)aminomethane is an organic compound with the formula (HOCH2)3CNH2. Synonyms are TRIS, Tris, Tris base, Tris buffer, Trizma, Trisamine, THAM, Tromethamine, Trometamol, Tromethane, Trisaminol. The preferred IUPAC name is 2-Amino-2-(hydroxymethyl) propane-1,3-diol. CAS Registry Number: 77-86-1.

The term “isoelectric point (pl)” is the pH at which a particular molecule or surface carries no net electrical charge. The pl of a polypeptide is dependent on the amino acids that make up the polypeptide. At a pH below its pl, the polypeptide carries a net positive charge. At a pH above its pl, the polypeptide carries a net negative charge. A polypeptide can therefore be separated on the basis of its ionization status at a given pH. The actual pl of a polypeptide can be affected by factors such as post-translational modification. The actual pl can be determined by experimental methods such as isoelectric focusing.

The terms “chromatography” refers to any current or future chromatography-based process of purifying one or more target molecules from a sample, e.g. by the removal of impurities and/or other non-target molecules. During chromatography a solute of interest, for example a polypeptide, in a mixture is separated from other solutes in a mixture as a result of differences in rates at which the individual solutes of the mixture migrate through a stationary medium under the influence of a moving phase, or in bind and elute processes. Examples of liquid chromatography purification include, but are not limited to: affinity chromatography, immobilized metal ion affinity chromatography flow-through chromatography, ion exchange chromatography, size-exclusion chromatography, reversed-phase chromatography, simulated moving-bed chromatography, hydrophobic interaction chromatography, gel filtration, chromato-focusing.

The term “mixed-mode chromatography” or “multimodal chromatography” refers to a purification process using mixed-mode adsorbents, which provide multiple modes of interaction, such as hydrophobic, cation exchange, and hydrogen bonding interaction between the polypeptide of interest and the adsorbent ligands. Commercially available mixed mode chromatography resins include Capto™ MMC, Capto™ MMC ImpRes, Capto Blue, Blue Sepharose™ 6 Fast Flow, Capto™ Adhere, and Capto™ Adhere ImpRes from GE Healthcare Life Sciences or Eshmuno® HCX from EMD Millipore, or Nuvia™ cPrime from Bio-Rad.

The terms “cation exchange resin,” “cation exchange adsorbent,” or “cation exchange matrix” refer to a solid phase which is negatively charged, and which thus has free cations for exchange with cations in an aqueous solution passed over or through the solid phase. A negatively charged ligand attached to the solid phase to form the cation exchange resin may be, e.g. a carboxylate or sulfonate. Commercially available cation exchange resins include carboxy-methyl-cellulose, sulphopropyl (SP) immobilized on agarose (e.g. SP Sepharose™ XL, SP-Sepharose™ Fast Flow, SP Sepharose™ High Performance, CM Sepharose™ Fast Flow, CM Sepharose™ High Performance, Capto™ S, and Capto™ SP ImpRes from GE Healthcare Life Sciences, or Fractogel® EMD SE HiCap, Fractogel® EMD SO3″, Fractogel® EMD COO″, Eshmuno™ S, and Eshmuno™ CPX from EMD Millipore, or UNOsphere™ S and Nuvia™ S from Bio-Rad).

The terms “anion exchange resin,” “anion exchange adsorbent,” or “anion exchange matrix” are used herein to refer to a solid phase, which is positively charged, e.g. having one or more positively charged ligands, such as quaternary amino groups, attached thereto. Commercially available anion exchange resins include DEAE Sepharose™ Fast Flow, Q Sepharose™ Fast Flow, Q Sepharose™ High Performance, Q Sepharose™ XL, Capto™ DEAE, Capto™ Q, and Capto™ Q ImpRes from GE Healthcare Life Sciences, or Fractogel® EMD TMAE HiCap, Fractogel® EMD DEAE, and Eshmuno Q from EMD Millipore, or U Osphere™ Q and Nuvia™ Q from Bio-Rad.

The term “antibody” refers to glycosylated and non-glycosylated immunoglobulins of any of the five major classes (isotypes) of immunoglobulins: IgA, IgD, IgE, IgG, and IgM, or subclasses thereof (e.g., IgG1, IgG2, IgG3, IgG4, lgA1 and IgA2) and combinations and variants thereof. As used herein, the term encompasses antibodies form any species (e.g. murine, canine, feline, IgY, etc.) and combinations thereof, e.g. human, humanized, chimeric antibodies. The term refers to monoclonal and polyclonal antibodies as well as to monospecific and multi-specific antibodies (such as bispecific antibodies). As used herein, the term also encompasses fusion proteins comprising an antigen determination portion, and any other modified immunoglobulin molecule comprising an antigen recognition site. As used herein, the term “antibody” includes intact immunoglobulins as well as antibody fragments, that refer to one or more portions of an antibody that retain the ability to specifically interact with (e.g., by binding, steric hindrance, stabilizing spatial distribution) an antigen. Examples of binding fragments include, but are not limited to Fab, Fab′, F(ab′) 2, Fd, Fv and dAb fragments (Ward et al., (1989) Nature 341:544-546), single chain Fv (scFv) (e.g., Bird et al., (1988) Science 242:423-426; and Huston et al., (1988) Proc. Natl. Acad. Sci. 85:5879-5883). Any naturally occurring, enzymatically obtainable, synthetic, alternative scaffold, or genetically engineered polypeptide that specifically binds an antigen are intended to be encompassed within the term “antibody” as used herein.

The terms “contaminant” and “impurity” are used interchangeably herein and refer to any objectionable molecule, including a biological macromolecule such as a DNA, an RNA, one or more host cell proteins, endotoxins, lipids and one or more additives which may be present in a sample containing the target protein that is being separated from one or more of the foreign or objectionable molecules using a process of the present invention. Additionally, such a contaminant may include any reagent, which is used in a step that may occur prior to the purification process.

“High molecular weight (HMW) species” include species having a higher molecular weight than the target protein mass, such as multimers. Multimers include everything other than the monomer of the target protein. For instance, a monomer of an IgG antibody encompasses the traditional tetrameric antibody composition comprising two heavy and light chains. Multimers include species having a higher molecular mass than the target protein mass, such as dimers (two identical proteins associated covalently or non-covalently) and aggregates (covalent or non-covalently associated whole and/or partial proteins).

“Low molecular weight (LMW) species” include species having a lower molecular weight than the target protein mass, such as clips, and degraded product.

As used herein, the term “polishing” refers to a downstream processing step which occurs after the initial (affinity) capture step and which is intended to remove residual amounts of impurities that are present in the product stream and which typically have more similarity to the product than the impurities removed during the capture step.

Methods for the determination of yield or purity of a polypeptide are known to those of skill in the art. Yield or purity of a polypeptide may be determined by any suitable method of analysis (e.g., band intensity on a silver stained gel, polyacrylamide gel electrophoresis, ELISA, HPLC and the like). An exemplary method is size-exclusion chromatography (SEC) high-performance liquid chromatography (HPLC). Purity may be determined using relative “area under the curve” (AUC) values, which can typically be obtained for peaks in a chromatogram, such as an HPLC chromatogram.

The term “bind and elute mode” refers to a product separation technique in which at least one product contained in a sample (e.g., an Fc region containing protein) binds to a chromatographic resin or media and is subsequently eluted.

The term “flow-through mode” refers to conditions at which the target protein will flow through while contaminants will bind to the chromatography support.

The amino acid and encoding nucleic acid sequences in Table 1 are an example of an IL-17C antibody, as well as portions thereof.

TABLE 1
Exemplary IL-17C antibody sequences

| Antibody | | SEQ ID No.: | [aa]/[DNA] |
|---|---|---|---|
| MAB#1 | HCDR1 | SEQ ID | DYAMH |
| | | No.: 1 | |
| | HCDR2 | SEQ ID | YIGGVGEGTQYAESVKG |
| | | No.: 2 | |
| | HCDR3 | SEQ ID | GFAIRYYGFDY |
| | | No.: 3 | |
| | LCDR1 | SEQ ID | SGDKLGDKYAY |
| | | No.: 4 | |
| | LCDR2 | SEQ ID | QDSKRPS |
| | | No.: 5 | |
| | LCDR3 | SEQ ID | QVFTFPLVTT |
| | | No.: 6 | |
| | VL | SEQ ID | SYELTQPPSVSVSPGQTASITCSGDKLGDKYAYWYQQKP |
| | | No.: 7 | GQSPVLVIYQDSKRPSGIPERFSGSNSGNTATLTISGTQAE |
| | | | DEADYYCQVFTFPLVTTVFGGGTKLTVLGQ |
| | VH | SEQ ID | EVQLLESGGGLVQPGGSLRLSCAASGFTVSDYAMHWVR |
| | | No.: 8 | QAPGKGLEWVSYIGGVGEGTQYAESVKGRFTISRDNSKN |
| | | | TLYLQMNSLRAEDTAVYYCARGFAIRYYGFDYWGQGTLV |
| | | | TVSS |
| | Light chain | SEQ ID | SYELTQPPSVSVSPGQTASITCSGDKLGDKYAYWYQQKP |
| | | No.: 9 | GQSPVLVIYQDSKRPSGIPERFSGSNSGNTATLTISGTQAE |
| | | | DEADYYCQVFTFPLVTTVFGGGTKLTVLGQPKAAPSVTLF |
| | | | PPSSEELQANKATLVCLISDFYPGAVTVAWKADSSPVKAG |
| | | | VETTTPSKQSNNKYAASSYLSLTPEQWKSHRSYSCQVTH |
| | | | EGSTVEKTVAPTECS |
| | Heavy | SEQ ID | EVQLLESGGGLVQPGGSLRLSCAASGFTVSDYAMHWVR |
| | chain | No.: 10 | QAPGKGLEWVSYIGGVGEGTQYAESVKGRFTISRDNSKN |
| | (IgG1) | | TLYLQMNSLRAEDTAVYYCARGFAIRYYGFDYWGQGTLV |
| | | | TVSSASTKGPSVFPLAPSSKSTSGGTAALGCLVKDYFPEP |
| | | | VTVSWNSGALTSGVHTFPAVLQSSGLYSLSSVVTVPSSSL |
| | | | GTQTYICNVNHKPSNTKVDKRVEPKSCDKTHTCPPCPAP |
| | | | ELLGGPSVFLFPPKPKDTLMISRTPEVTCVVVDVSHEDPE |
| | | | VKFNWYVDGVEVHNAKTKPREEQYNSTYRVVSVLTVLHQ |
| | | | DWLNGKEYKCKVSNKALPAPIEKTISKAKGQPREPQVYTL |
| | | | PPSREEMTKNQVSLTCLVKGFYPSDIAVEWESNGQPENN |
| | | | YKTTPPVLDSDGSFFLYSKLTVDKSRWQQGNVFSCSVMH |
| | | | EALHNHYTQKSLSLSPGK |
| | VL | SEQ ID | TCCTACGAGCTGACCCAGCCCCCCTCCGTGTCCGTGTC |
| | | No.: 11 | TCCTGGCCAGACCGCCTCCATCACCTGTTCCGGCGACA |
| | | | AGCTGGGCGATAAGTACGCCTACTGGTATCAGCAGAAG |
| | | | CCCGGCCAGTCCCCCGTGCTGGTCATCTACCAGGACT |
| | | | CCAAGCGGCCCTCCGGCATCCCTGAGCGGTTCTCCGG |
| | | | CTCCAACTCCGGCAACACCGCCACCCTGACCATCTCCG |
| | | | GCACCCAGGCCGAGGACGAGGCCGACTACTACTGCCA |
| | | | GGTGTTCACCTTCCCCCTGGTCACCACCGTGTTCGGCG |
| | | | GAGGCACCAAGCTGACCGTGCTGGGCCAG |
| | VH | SEQ ID | GAGGTGCAGCTGCTGGAATCCGGCGGAGGACTGGTGC |
| | | No.: 12 | AGCCTGGCGGCTCCCTGAGACTGTCTTGCGCCGCCTC |
| | | | CGGCTTCACCGTGTCCGACTACGCTATGCACTGGGTCC |
| | | | GACAGGCCCCTGGCAAGGGCCTGGAATGGGTGTCCTA |
| | | | TATCGGCGGCGTGGGCGAGGGCACCCAGTACGCTGAG |
| | | | TCTGTGAAGGGCCGGTTCACCATCTCCCGGGACAACTC |
| | | | CAAGAACACCCTGTACCTGCAGATGAACTCCCTGCGGG |
| | | | CCGAGGACACCGCCGTGTACTACTGTGCCAGAGGCTT |
| | | | CGCCATCCGGTACTACGGCTTCGACTACTGGGGCCAG |
| | | | GGCACCCTGGTCACCGTGTCTAGC |
| | Light chain | SEQ ID | TCCTACGAGCTGACCCAGCCCCCCTCCGTGTCCGTGTC |
| | | No.: 13 | TCCTGGCCAGACCGCCTCCATCACCTGTTCCGGCGACA |
| | | | AGCTGGGCGATAAGTACGCCTACTGGTATCAGCAGAAG |
| | | | CCCGGCCAGTCCCCCGTGCTGGTCATCTACCAGGACT |
| | | | CCAAGCGGCCCTCCGGCATCCCTGAGCGGTTCTCCGG |
| | | | CTCCAACTCCGGCAACACCGCCACCCTGACCATCTCCG |
| | | | GCACCCAGGCCGAGGACGAGGCCGACTACTACTGCCA |
| | | | GGTGTTCACCTTCCCCCTGGTCACCACCGTGTTCGGCG |
| | | | GAGGCACCAAGCTGACCGTGCTGGGCCAGCCTAAGGC |
| | | | CGCTCCCTCCGTGACCCTGTTCCCCCCATCCTCCGAGG |
| | | | AACTGCAGGCCAACAAGGCCACCCTGGTCTGCCTGATC |
| | | | TCCGACTTCTACCCTGGCGCCGTGACCGTGGCCTGGA |
| | | | AGGCCGACAGCTCTCCTGTGAAGGCCGGCGTGGAAAC |
| | | | CACCACCCCCTCCAAGCAGTCCAACAACAAATACGCCG |
| | | | CCTCCTCCTACCTGTCCCTGACCCCCGAGCAGTGGAAG |
| | | | TCCCACCGGTCCTACAGCTGCCAGGTCACACACGAGG |
| | | | GCTCCACCGTGGAAAAGACCGTGGCCCCTACCGAGTG |
| | | | CTCC |
| | Heavy | SEQ ID | GAGGTGCAGCTGCTGGAATCCGGCGGAGGACTGGTGC |
| | chain | No.: 14 | AGCCTGGCGGCTCCCTGAGACTGTCTTGCGCCGCCTC |
| | (IgG1) | | CGGCTTCACCGTGTCCGACTACGCTATGCACTGGGTCC |
| | | | GACAGGCCCCTGGCAAGGGCCTGGAATGGGTGTCCTA |
| | | | TATCGGCGGCGTGGGCGAGGGCACCCAGTACGCTGAG |
| | | | TCTGTGAAGGGCCGGTTCACCATCTCCCGGGACAACTC |
| | | | CAAGAACACCCTGTACCTGCAGATGAACTCCCTGCGGG |
| | | | CCGAGGACACCGCCGTGTACTACTGTGCCAGAGGCTT |
| | | | CGCCATCCGGTACTACGGCTTCGACTACTGGGGCCAG |
| | | | GGCACCCTGGTCACCGTGTCTAGCGCCTCCACCAAGG |
| | | | GCCCCTCCGTGTTCCCTCTGGCCCCCTCCAGCAAGTCC |
| | | | ACCTCTGGCGGCACCGCTGCCCTGGGCTGCCTGGTCA |
| | | | AGGACTACTTCCCCGAGCCCGTGACCGTGTCCTGGAAC |
| | | | TCTGGCGCCCTGACCTCCGGCGTGCACACCTTCCCTG |
| | | | CCGTGCTGCAGTCCTCCGGCCTGTACTCCCTGTCCTCC |
| | | | GTCGTGACCGTGCCCTCCAGCTCTCTGGGCACCCAGA |
| | | | CCTACATCTGCAACGTGAACCACAAGCCCTCCAACACC |
| | | | AAGGTGGACAAGCGGGTGGAACCCAAGTCCTGCGACA |
| | | | AGACCCACACCTGTCCCCCCTGCCCTGCCCCTGAACTG |
| | | | CTGGGCGGACCTTCCGTGTTCCTGTTCCCCCCAAAGCC |
| | | | CAAGGACACCCTGATGATCTCCCGGACCCCCGAAGTGA |
| | | | CCTGCGTGGTGGTGGACGTGTCCCACGAGGACCCTGA |
| | | | AGTGAAGTTCAATTGGTACGTGGACGGCGTGGAAGTGC |
| | | | ACAACGCCAAGACCAAGCCCAGAGAGGAACAGTACAAC |
| | | | TCCACCTACCGGGTGGTGTCCGTGCTGACCGTGCTGC |
| | | | ACCAGGACTGGCTGAACGGCAAAGAGTACAAGTGCAA |
| | | | GGTGTCCAACAAGGCCCTGCCTGCCCCCATCGAAAAGA |
| | | | CCATCTCCAAGGCCAAGGGCCAGCCCCGCGAGCCCCA |
| | | | GGTGTACACACTGCCCCCTAGCCGGGAAGAGATGACC |
| | | | AAGAACCAGGTGTCCCTGACCTGTCTGGTCAAGGGCTT |
| | | | CTACCCCTCCGACATTGCCGTGGAATGGGAGTCCAACG |
| | | | GCCAGCCCGAGAACAACTACAAGACCACCCCCCCTGT |
| | | | GCTGGACTCCGACGGCTCATTCTTCCTGTACTCCAAGC |
| | | | TGACCGTGGACAAGTCCCGGTGGCAGCAGGGCAACGT |
| | | | GTTCTCCTGCTCCGTGATGCACGAGGCCCTGCACAACC |
| | | | ACTACACCCAGAAGTCCCTGTCCCTGAGCCCCGGCAAG |

### WORKING EXAMPLES

### Example 1

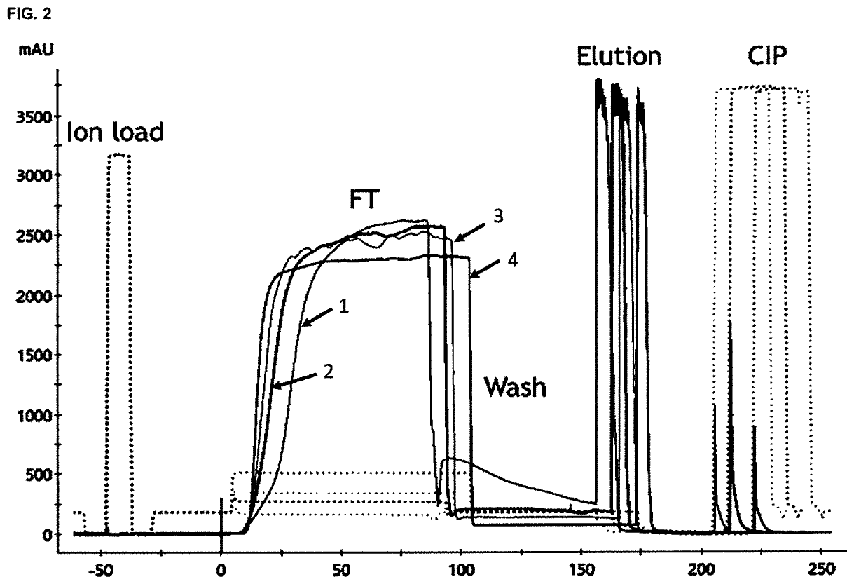

To test the impact of Tris addition (i.e. increasing Tris concentration) to a sample comprising an antibody with heavy chain of SEQ ID NO.: 10 and light chain of SEQ ID NO.: 9 prior to loading a Capto adhere ImpRes column (GE Healthcare) in flow-through mode on yield and purity, four different purification runs were carried out. One run without Tris addition and three runs with addition of 5, 10 and 20% (v/v) of 2 M Tris pH 7.1 to the sample eluate. Antibody yield and purity (SEC monomer) of the resulting flow-throughs were analyzed. Results are listed in Table 2 and corresponding chromatograms are shown as overlay in FIG. 2. Addition of 5% (v/v) Tris led to an increase in yield of 20% with only 0.4% decreased SEC monomer portion of the resulting pool. Increased quantities of Tris added to the sample resulted in further increased yield, however to a lesser extent (˜4% and ˜7% increase compared to the 5% Tris addition for 10% and 20% Tris addition, respectively) and further slight decrease of monomer portion.

TABLE 2
Yield and monomer content of the antibody in the
flow-through after multimodal AEX chromatography
using Capto ® adhere ImpRes (GE Healthcare)

| | 2M Tris pH 7.1 [% (v/v)] | | | |
|---|---|---|---|---|
| | none | 5 | 10 | 20 |
| Conductivity [mS/cm] | 8.2 | 13.6 | 17.2 | 25.5 |
| Yield [%] | 58.0 | 78.0 | 81.9 | 84.6 |
| SEC Monomer [%] | 97.8 | 97.4 | 97.0 | 96.6 |

### Example 2

To elucidate, that the addition of Tris to the antibody sample as in Example 1, and not only the adjustment of conductivity, improves antibody yield in the flow through after Capto adhere ImpRes chromatography a head-to-head comparison between pre-conditioning of the sample with Tris (run 1) and pre-conditioning of the sample with NaCl (run 2) was performed. For sample preparation, conductivity was adjusted in run 1 with 2 M Tris pH 7.1 and in run 2 with 5 M NaCl to a target conductivity of 15 mS/cm (Table 3).

TABLE 3

| | | | Conductivity | | |
|---|---|---|---|---|---|
| | PH | | Start | Target | |
| Run | Start | Target | [mS/cm] | [mS/cm] | Comment |
| 1 | 5.50 | 7.10 | 6.80 | 15.00 | Conductivity adjusted |
| | | | | | with 2M Tris pH 7.1 |
| 2 | 5.49 | 7.10 | 7.00 | 15.00 | Conductivity adjusted |
| | | | | | with 5M NaCl |

Both loads have the same conductivity but differ in their buffer matrix. pH and conductivity measurements were performed at ambient temperature 20° C. ±2° C. Purification results and QC data are shown in Table 4.

TABLE 4

| | | SEC/MALS | | | | | |
|---|---|---|---|---|---|---|---|
| | | | HMW | LMW | Yield after | Recovery | |
| | | Monomer | impurities | impurities | Purification | (rel. to | |
| Run | Mode | [%] | [%] | [%] | [mg] | Load) [%] | |
| 1 | MM-AIEX | 96.7 | 2.1 | 1.1 | 675.2 | 81.5% | conductivity |
| | | | | | | | adjusted with |
| | | | | | | | 2M Tris pH 7.1 |
| 2 | MM-AIEX | 97.1 | 1.7 | 1.2 | 644.6 | 77.3% | conductivity |
| | | | | | | | adjusted with |
| | | | | | | | 5M NaCl |

Without addition of Tris (run 2), the yield is about 5% less compared to run 1, in which conductivity was adjusted to 15 mS/cm with 2 M Tris, PH 7.1.

1. A method of producing an antibody composition, comprising purifying an IgG1 antibody by a process comprising the steps of:
a) providing a sample with a first pH comprising the antibody;
b) adjusting the first pH of the sample to a second pH;
c) adjusting the conductivity of the sample and the second pH to a third pH;
d) processing the adjusted sample by ion exchange chromatography; and
e) collecting the antibody in a composition,
wherein the pH of the sample in step b) and/or the pH and conductivity of the sample in step c) is adjusted with Tris in the absence of a salt.

2. The method of claim 1, wherein the pH of the sample in step b) and the pH and conductivity of the sample in step c) are adjusted with Tris in the absence of the salt.

3. The method of claim 1, wherein the collected antibody has a SEC monomer content of at least 98%.

4. The method of claim 1, wherein the collected antibody has a SEC monomer content of at least 99%.

5. The method of claim 1, wherein the processing the adjusted sample by ion exchange chromatography results in an antibody yield of at least 75%.

6. The method of claim 1, wherein the collected antibody has a SEC monomer content of at least 98%, and the processing the adjusted sample by ion exchange chromatography results in an antibody yield of at least 75%.

7. The method of claim 6, wherein the pH of the composition comprising the antibody collected in step e) is adjusted using an acetate buffer.

8. The method of claim 1, wherein the antibody is a human monoclonal antibody.

9. The method of claim 8, wherein the antibody is an anti-IL-17 antibody.

10. The method of claim 1, wherein the ion exchange chromatography is anion exchange chromatography.

11. The method of claim 1, wherein the ion exchange chromatography is performed in bind-and-elute mode.

12. The method of claim 1, wherein the second pH is about pH 5.2 to about pH 5.6.

13. The method of claim 8, wherein the collected antibody has a SEC monomer content of at least 98%, and the processing the adjusted sample by ion exchange chromatography results in an antibody yield of at least 75%.

14. A method of producing an antibody composition, comprising purifying an IgG1 antibody by a process comprising the steps of:
a) providing a sample with a first pH comprising the antibody;
b) adjusting the first pH of the sample to a second pH;
c) adjusting the conductivity of the sample and the second pH to a third pH;
d) processing the adjusted sample by ion exchange chromatography; and
e) collecting the antibody,
wherein the pH of the sample in step b) and/or the pH and conductivity of the sample in step c) is adjusted with Tris in the absence of NaCl.

15. The method of claim 14, wherein the antibody is an anti-IL-17 antibody.

16. The method of claim 14, wherein the collected antibody has a SEC monomer content of at least 98%.

17. The method of claim 14, wherein the processing the adjusted sample by ion exchange chromatography results in an antibody yield of at least 75%.

18. The method of claim 14, wherein the collected antibody has a SEC monomer content of at least 98%, and the processing the adjusted sample by ion exchange chromatography results in an antibody yield of at least 75%.

19. The method of claim 18, wherein the pH of the composition comprising the antibody collected in step e) is adjusted using an acetate buffer.

20. A method of producing an antibody composition, comprising purifying an anti-IL-17 IgG1 antibody by a process comprising the steps of:
a) providing a sample with a first pH comprising the antibody;
b) adjusting the first pH of the sample to a second pH;
c) adjusting the conductivity of the sample and the second pH to a third pH;
d) processing the adjusted sample by ion exchange chromatography; and
e) collecting the antibody,
wherein the pH and conductivity of the sample in step c) is adjusted with Tris in the absence of NaCl.

21. The method of claim 20, wherein the pH of the sample in step b) is adjusted with Tris in the absence of NaCl.

22. The method of claim 20, wherein the collected antibody has a SEC monomer content of at least 98%.

23. The method of claim 20, wherein the processing the adjusted sample by ion exchange chromatography results in an antibody yield of at least 75%.

24. The method of claim 20, wherein the collected antibody has a SEC monomer content of at least 98%, and the processing the adjusted sample by ion exchange chromatography results in an antibody yield of at least 75%.

25. The method of claim 24, wherein the pH of the composition comprising the antibody collected in step e) is adjusted using an acetate buffer.

26. The method of claim 24, wherein the ion exchange chromatography is anion exchange chromatography.

27. The method of claim 25, wherein the ion exchange chromatography is performed in bind-and-elute mode.

28. A pharmaceutical composition produced by the method of claim 1.

29. A pharmaceutical composition produced by the method of claim 15.

30. A pharmaceutical composition produced by the method of claim 20.


SEQUENCE LISTING (14 sequences)

SEQ ID NO: 1 - protein, 5 aa, synthetic construct
  REGION 1..5: note = source = /note="Description of Artificial Sequence: Synthetic peptide"
DYAMH

SEQ ID NO: 2 - protein, 17 aa, synthetic construct
  REGION 1..17: note = source = /note="Description of Artificial Sequence: Synthetic peptide"
YIGGVGEGTQYAESVKG

SEQ ID NO: 3 - protein, 11 aa, synthetic construct
  REGION 1..11: note = source = /note="Description of Artificial Sequence: Synthetic peptide"
GFAIRYYGFDY

SEQ ID NO: 4 - protein, 11 aa, synthetic construct
  REGION 1..11: note = source = /note="Description of Artificial Sequence: Synthetic peptide"
SGDKLGDKYAY

SEQ ID NO: 5 - protein, 7 aa, synthetic construct
  REGION 1..7: note = source = /note="Description of Artificial Sequence: Synthetic peptide"
QDSKRPS

SEQ ID NO: 6 - protein, 10 aa, synthetic construct
  REGION 1..10: note = source = /note="Description of Artificial Sequence: Synthetic peptide"
QVFTFPLVTT

SEQ ID NO: 7 - protein, 110 aa, synthetic construct
  REGION 1..110: note = source = /note="Description of Artificial Sequence: Synthetic polypeptide"
SYELTQPPSVSVSPGQTASITCSGDKLGDKYAYWYQQKPGQSPVLVIYQDSKRPSGIPERFSGSNSGNTATLTISGTQAEDEADYYCQVFTFPLVTTVFGGGTKLTVLGQ

SEQ ID NO: 8 - protein, 120 aa, synthetic construct
  REGION 1..120: note = source = /note="Description of Artificial Sequence: Synthetic polypeptide"
EVQLLESGGGLVQPGGSLRLSCAASGFTVSDYAMHWVRQAPGKGLEWVSYIGGVGEGTQYAESVKGRFTISRDNSKNTLYLQMNSLRAEDTAVYYCARGFAIRYYGFDYWGQGTLVTVSS

SEQ ID NO: 9 - protein, 214 aa, synthetic construct
  REGION 1..214: note = source = /note="Description of Artificial Sequence: Synthetic polypeptide"
SYELTQPPSVSVSPGQTASITCSGDKLGDKYAYWYQQKPGQSPVLVIYQDSKRPSGIPERFSGSNSGNTATLTISGTQAEDEADYYCQVFTFPLVTTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 450 aa, synthetic construct
  REGION 1..450: note = source = /note="Description of Artificial Sequence: Synthetic polypeptide"
EVQLLESGGGLVQPGGSLRLSCAASGFTVSDYAMHWVRQAPGKGLEWVSYIGGVGEGTQYAESVKGRFTISRDNSKNTLYLQMNSLRAEDTAVYYCARGFAIRYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 330 bp, synthetic construct
  misc_feature 1..330: note = source = /note="Description of Artificial Sequence: Synthetic polynucleotide"
  source 1..330: mol_type = other DNA
tcctacgagctgacccagcccccctccgtgtccgtgtctcctggccagaccgcctccatcacctgttccggcgacaagctgggcgataagtacgcctactggtatcagcagaagcccggccagtcccccgtgctggtcatctaccaggactccaagcggccctccggcatccctgagcggttctccggctccaactccggcaacaccgccaccctgaccatctccggcacccaggccgaggacgaggccgactactactgccaggtgttcaccttccccctggtcaccaccgtgttcggcggaggcaccaagctgaccgtgctgggccag

SEQ ID NO: 12 - DNA, 360 bp, synthetic construct
  misc_feature 1..360: note = source = /note="Description of Artificial Sequence: Synthetic polynucleotide"
  source 1..360: mol_type = other DNA
gaggtgcagctgctggaatccggcggaggactggtgcagcctggcggctccctgagactgtcttgcgccgcctccggcttcaccgtgtccgactacgctatgcactgggtccgacaggcccctggcaagggcctggaatgggtgtcctatatcggcggcgtgggcgagggcacccagtacgctgagtctgtgaagggccggttcaccatctcccgggacaactccaagaacaccctgtacctgcagatgaactccctgcgggccgaggacaccgccgtgtactactgtgccagaggcttcgccatccggtactacggcttcgactactggggccagggcaccctggtcaccgtgtctagc

SEQ ID NO: 13 - DNA, 642 bp, synthetic construct
  misc_feature 1..642: note = source = /note="Description of Artificial Sequence: Synthetic polynucleotide"
  source 1..642: mol_type = other DNA
tcctacgagctgacccagcccccctccgtgtccgtgtctcctggccagaccgcctccatcacctgttccggcgacaagctgggcgataagtacgcctactggtatcagcagaagcccggccagtcccccgtgctggtcatctaccaggactccaagcggccctccggcatccctgagcggttctccggctccaactccggcaacaccgccaccctgaccatctccggcacccaggccgaggacgaggccgactactactgccaggtgttcaccttccccctggtcaccaccgtgttcggcggaggcaccaagctgaccgtgctgggccagcctaaggccgctccctccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaggtcacacacgagggctccaccgtggaaaagaccgtggcccctaccgagtgctcc

SEQ ID NO: 14 - DNA, 1350 bp, synthetic construct
  misc_feature 1..1350: note = source = /note="Description of Artificial Sequence: Synthetic polynucleotide"
  source 1..1350: mol_type = other DNA
gaggtgcagctgctggaatccggcggaggactggtgcagcctggcggctccctgagactgtcttgcgccgcctccggcttcaccgtgtccgactacgctatgcactgggtccgacaggcccctggcaagggcctggaatgggtgtcctatatcggcggcgtgggcgagggcacccagtacgctgagtctgtgaagggccggttcaccatctcccgggacaactccaagaacaccctgtacctgcagatgaactccctgcgggccgaggacaccgccgtgtactactgtgccagaggcttcgccatccggtactacggcttcgactactggggccagggcaccctggtcaccgtgtctagcgcctccaccaagggcccctccgtgttccctctggccccctccagcaagtccacctctggcggcaccgctgccctgggctgcctggtcaaggactacttccccgagcccgtgaccgtgtcctggaactctggcgccctgacctccggcgtgcacaccttccctgccgtgctgcagtcctccggcctgtactccctgtcctccgtcgtgaccgtgccctccagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtgtccaacaaggccctgcctgcccccatcgaaaagaccatctccaaggccaagggccagccccgcgagccccaggtgtacacactgccccctagccgggaagagatgaccaagaaccaggtgtccctgacctgtctggtcaagggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag